(19) United States
(12) Patent Application Publication
(10) Pub. No.: US 2010/0280471 A1
(43) Pub. Date: Nov. 4, 2010

(54) CHANNELED APERTURED FILM AND PERSONAL CARE ARTICLES CONTAINING THE FILM

(75) Inventor: Ashok Harakhlal Shah, Midlothian, VA (US)

Correspondence Address: NAVS, LLC, 14100 Southshore Road, Midlothian, VA, 23112, US

(73) Assignee: NAVS, LLC, Midlothian, VA (US)

(21) Appl. No.: 12/432,007

(22) Filed: Apr. 29, 2009

(51) Int. Cl. A61F 13/15; B32B 3/24; B29C 59/04

(52) U.S. Cl. 604/367; 428/131; 264/284; 264/156; 604/385.23

(57) ABSTRACT

An absorbent personal care article comprising an apertured polymer film having a first series of channels, each having a length, width and depth, on a first surface thereof and apertures having diameters at the bottoms of and along the length of said channels, said apertures extending through the thickness of the film and through a second surface of said film; and an absorbent core disposed against said second surface of said apertured film.

### FIELD

This invention relates to a novel apertured film having channels for air and moisture movement along the surface of the film. The novel apertured film can find use in personal care articles when disposed in the space between the adjacent body skin and an absorbent layer of the personal care articles.

### BACKGROUND

The purpose of the personal care absorbent articles, such as, diapers, incontinence garments, training pants, catamenial pads, panty liners and bandages, is to absorb and contain body exudates like urine, blood, menses and feces. Personal care absorbent articles typically include a body side liner (also known as a top sheet) adapted to be placed adjacent to the wearer's skin, a back side layer which is usually liquid impervious and an absorbent core in the middle for receiving and retaining exuded body fluids. Some of the most important parameters for such articles are fluid intake time, rewet and reduced surface run-off. In addition, the top sheet and overall article needs to be soft to body contact.

Personal care absorbent articles basically move the liquid exudate away from body skin and into the storage retention absorbent core beneath the top sheet. The liquid exudate stored in the absorbent core and body heat create a very humid warm environment, which is ideal for bacteria growth, resulting in skin diseases to the body area exposed to such moist humid environment. One of the most well known of such diseases is diaper rash.

To be effective, personal care absorbent article must take in and transport the exuded body fluid to the absorbent core as quickly as possible. Once the body fluid is taken in, it is desirable that the fluid does not flow back toward and rewet the body side skin in contact with the top sheet of personal care absorbent article. By increasing the rate at which a fluid is taken into the absorbent core and by reducing the amount of rewet, the body skin will most likely be cleaner and free of body liquid exudate.

U.S. Pat. No. 5,702,382 and WO 93/09741, both of which are incorporated herein by reference in their entireties, describe the use of a nonwoven layer with an apertured film for reducing rewet and run-off. WO 93/09741 extensively describes an apertured film most commonly used in the trade, a nonwoven layer used outside and above the apertured film, and resins used in making the apertured film and nonwoven layer for desired softness and wetting characteristics, absorbent core and back sheet/layer.

U.S. Pat. No. 4,609,518, incorporated herein by reference in its entirety, describes equipment and processes involved in manufacturing individual components and finished composite (personal care absorbent articles of interest here).

Many disclosures are cited in these references covering details on ingredients and equipment used in manufacturing apertured films and nonwoven layers, including surface tension reducing polymer additives, finishing operations, application of wetting agents, thermal bonding, such as through-air bonding, adhesive bonding, etc.

U.S. Pat. No. 7,378,568 B2, incorporated herein by reference in its entirety, discloses an absorbent article having a topsheet and an absorbent core material. The acquisition distribution layer is located between the topsheet and the absorbent core material. The acquisition distribution layer is made of a three dimensional apertured film that defines a large void volume space between the acquisition distribution layer and the absorbent core material. The acquisition distribution layer provides high void volume for lateral spillage during repeated insult moments because the topsheet, which is in contact with the user, is held away from dispersing fluid that is unabsorbed by saturated core material. The void volume space provides a pathway for unabsorbed fluid to flow over the top plane of saturated core regions to unsaturated regions of the core material for absorption. The void volume space allows this migration of fluid to occur without the fluid coming into contact with the topsheet, thereby avoiding a feeling of wetness for a wearer.

U.S. Pat. No. 7,438,707 B2, incorporated herein by reference in its entirety, discusses the effect of warm humid environments on body skin in contact with such environment involved with personal care absorbent articles. The outer cover of the absorbent article per U.S. Pat. No. 7,438,707 B2 is differentially stretched upon receiving a load like liquid exudates, which causes differential movement of an absorbent core which is attached to the outer layer with respect to the top sheet, resulting in a gap between the top sheet (body side liner) and the absorbent core, facilitating the flow of air and vapor through the gap space between absorbent core and top sheet in a loaded condition of the absorbent core.

Although U.S. Pat. No. 7,438,707 B2 represents an improvement, it still falls short of addressing the space between the liquid exudate transporting apertured film layer and the skin, where the warm high humidity environment can thrive.

### SUMMARY

In a first embodiment, the present application is directed to an apertured film comprising a polymer film having a first series of channels, each having a length, width and depth, on at least one surface thereof and apertures having diameters at the bottoms of and along the length of said channels, said apertures extending through the thickness of the film, and wherein each aperture is disposed at the bottom of a channel.

In another embodiment, the application is directed to an apertured film as above, wherein the channels are straight.

In an alternative embodiment, the application is directed to an apertured film as above, wherein the channels are intersected by at least a second series of channels running at angles to said first series of channels.

Conveniently, the channels are curved.

In an alternative embodiment, the channels are sinusoidal.

In another embodiment, the application is directed to an apertured film as above, wherein the length of the channels is at least ten times their equivalent diameters, and the depths of the channels are between about 0.25 to about 1 times the diameters of the apertures.

In another embodiment, the application is directed to an apertured film as above, wherein the length of the channels is at least twenty times their equivalent diameters, and the depths of the channels are between about 0.5 to about 0.75 times the diameters of the apertures.

Conveniently, the width of the channels is at least the same as the diameters of the apertures.

Conveniently, the channels are continuous.

Conveniently, when at least two series of channels are running at angles to each other, both series of channels are continuous.

In one embodiment, the present application is directed to an apertured film, as described above, wherein the channels are disposed side-by-side.

Conveniently, the first surface of said apertured polymer film is hydrophilic and the second surface is hydrophobic.

In another embodiment, the present application is directed to an absorbent personal care article comprising an apertured polymer film having a first series of channels, each having a length, width and depth, on a first surface thereof and apertures having diameters at the bottoms of and along the length of said channels, wherein each aperture is disposed at the bottom of a channel, said apertures extending through the thickness of the film and through a second surface of said film; and an absorbent core disposed against said second surface of said apertured film.

In another embodiment, the absorbent personal care article further comprises a liquid pervious top layer of material, having first and second surfaces, and wherein said second surface is disposed against said first surface of said apertured polymer film.

Conveniently, the top layer material is a nonwoven material, which can be selected from the group consisting of a spunbonded webs, meltblown webs, air-laid webs, wet-laid webs, carded webs, hydroentangled webs and combinations thereof.

In another embodiment, the absorbent personal care article can be constructed such that the second surface of the top layer is attached to the first surface of the apertured film.

Conveniently, the second surface of the top layer is attached to the first surface of said apertured film using an adhesive applied in a series of lines having finite spacing and depth, forming channels.

The absorbent personal care article can further comprise a liquid impervious, moisture permeable back sheet disposed against the absorbent core opposite the apertured film.

In another embodiment of the absorbent personal care article, the first surface of the apertured polymer film is hydrophilic and the second surface is hydrophobic.

In another embodiment of the absorbent personal care article, the top layer material has a series of channels disposed on its second surface.

Conveniently, the absorbent personal care article of this application finds use in applications selected from the group consisting of diapers, incontinence garments, training pants, catamenial pads, panty liners and bandages.

In another embodiment, the present invention is directed to a process for forming an apertured film having channels on its surface and aperture holes at the bottom of said channels, comprising passing a film through a nip between male and female embossing rolls, wherein the female roll has engraved channels and apertures holes at the bottom of the engraved channels and the male embossing roll has raised wall patterns configured to correspond with said engraved channels.

Conveniently, process is further defined such that said film stays on the female embossing roll after passing through the nip and further comprises forming said aperture holes in the film by applying a pressure differential to the film at the sites of the aperture holes on the female embossing roll.

In another embodiment, the process is further defined such that the male embossing roll is porous to air flow under said pressure differential.

### BRIEF DESCRIPTION OF THE DRAWINGS

FIG. 1 shows the cross section of the absorbent article, showing top sheet, absorbent core and outer layer (backsheet).

FIG. 2 shows the novel apertured film where channels on the surface are predominantly running along one direction.

FIG. 3 shows the novel apertured film where channels on the surface are running in different directions.

FIGS. 4A and 4B show top view and cross section view, respectively, of the novel apertured film.

### DETAILED DESCRIPTION OF THE EMBODIMENTS

In response to the need for an effective means for reducing the potential for high humidity environments in the space adjacent to body skin, a novel top sheet is described herein. The novel top sheet comprises at least an apertured film, which has channels on its surface that faces the body side, optionally in combination with another top layer material, such as a nonwoven web. The channels have a finite depth, width and length for allowing liquid and air movement along the channels. The aperture holes are located at the base/bottom of the channels. In other words, the mouths of the aperture holes that receive body liquid exudate are located at the bottom of the channels.

The channels communicate to the exterior environment directly or through a moisture breathable outer layer when used in personal care absorbent article containing an absorbent core enclosed between at least an aperture film facing the body side and moisture breathable outer layer on the other side.

The apertured film as described herein can be used either alone or in combination with a nonwoven layer, for use as a top sheet for personal care absorbent articles coming in contact with body skin for an extended period. Examples of such articles are diapers, incontinence garments, training pants, catamenial pads, panty liners, bandages and the like.

Suitable polymers for forming the apertured films include polyethylene, such as LLDPE and LDPE, polypropylene, polyvinyl chloride, starch based resins, polyvinyl alcohol, polyurethanes, polycaprolactone and cellulose esters.

The channels can be straight along their lengths. However, it is advantageous if the channels are curved, e.g. sinusoidal, half circles, or Z-shaped along their lengths, for creating torturous paths for liquid exudate flowing through the channels along the surface of the apertured film, so that the liquid exudate is more likely to flow into the aperture holes at the bottoms of and along the lengths of the channels. Such controlled movement of liquid exudate along the lengths of the channels helps in distributing the liquid exudate over a larger, but managed area of the absorbent core underneath, rather than into a localized, concentrated area of the absorbent core. This helps in reducing the potential for liquid exudate flowing back through aperture holes, a phenomenon commonly known as re-wetting. Notably, there is only a single row of aperture holes along the length of and at the bottom of each channel.

The top side of the apertured film that receives the liquid exudate can be hydrophilic for rapid transport of the liquid exudate through the aperture holes. The hydrophilic characteristic is most commonly achieved by the use of wetting agents in the base resin formulation used in film manufacturing. In addition, the bottom surface of the apertured film in contact with the absorbent core can have hydrophobic surface characteristics (e.g., surface tension of a linear low density polyethylene (LLDPE) or low density polyethylene (LDPE) resin-based film without the use of wetting agent in the base resin; resins commonly used in manufacturing the apertured film). The hydrophobic characteristic of the bottom surface of the apertured film can be helpful in reducing accumulation of the free liquid exudate in the space between the bottom of the apertured film and the absorbent core. Due to the reduction in free liquid exudate under the apertured film, combined with the channels above the top of the aperture holes, the potential for back flow of liquid exudate through aperture holes and rewetting of the body skin is greatly reduced.

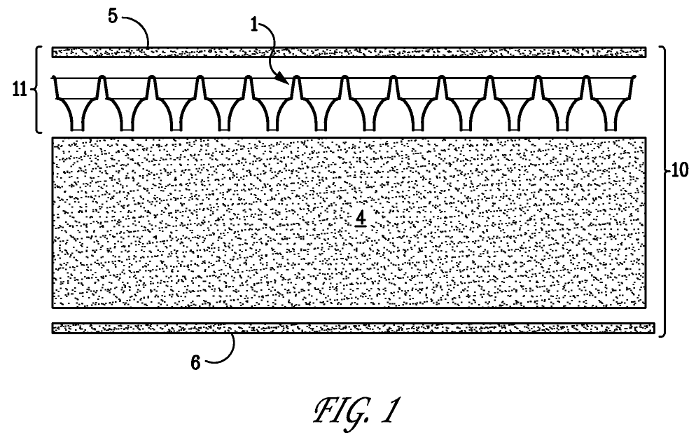

Personal care absorbent article 10 (FIG. 1) comprises absorbent core 4 enclosed by top sheet 11 on a top side and liquid impervious, moisture permeable backsheet film 6 on a bottom side (also termed as outer side). Suitable absorbents for the core include comminuted wood pulp which is generally referred to as airfelt, creped cellulose wadding, hydrogel-forming polymer gelling agents, modified cross-linked cellulose fibers, capillary channel fibers, absorbent foams, absorbent sponges, synthetic staple fibers, polymeric fibers, peat moss, or any equivalent material or combinations of materials.

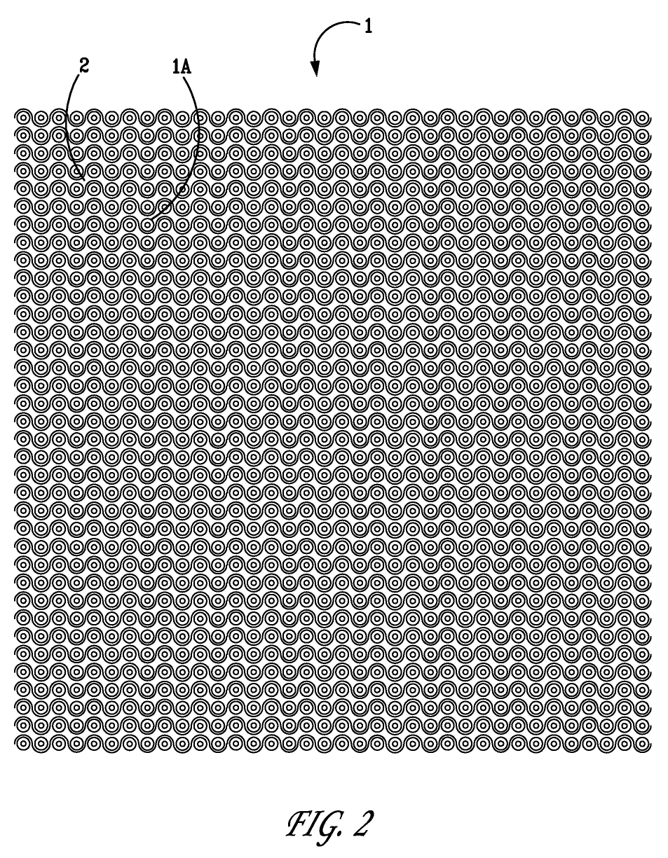

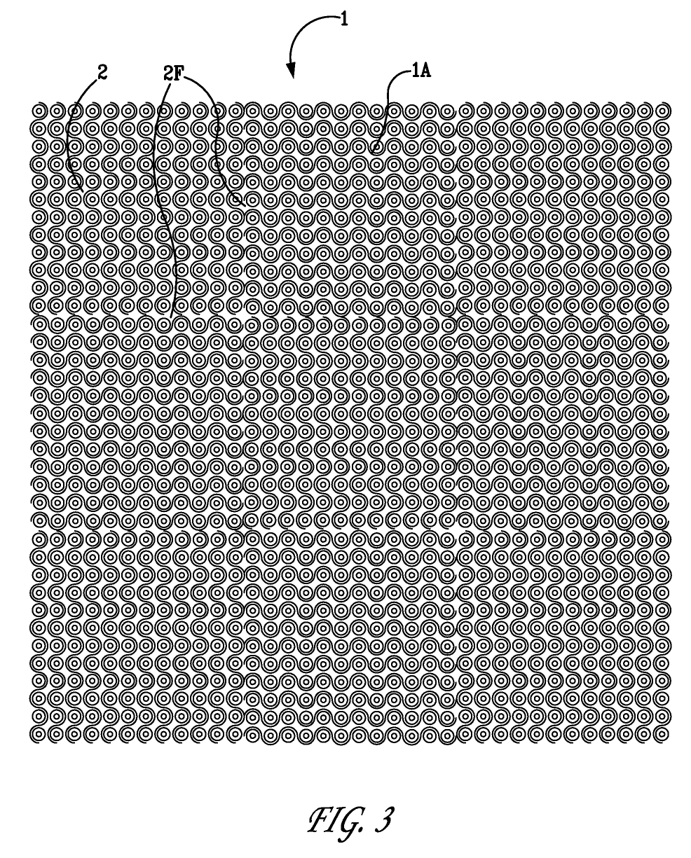

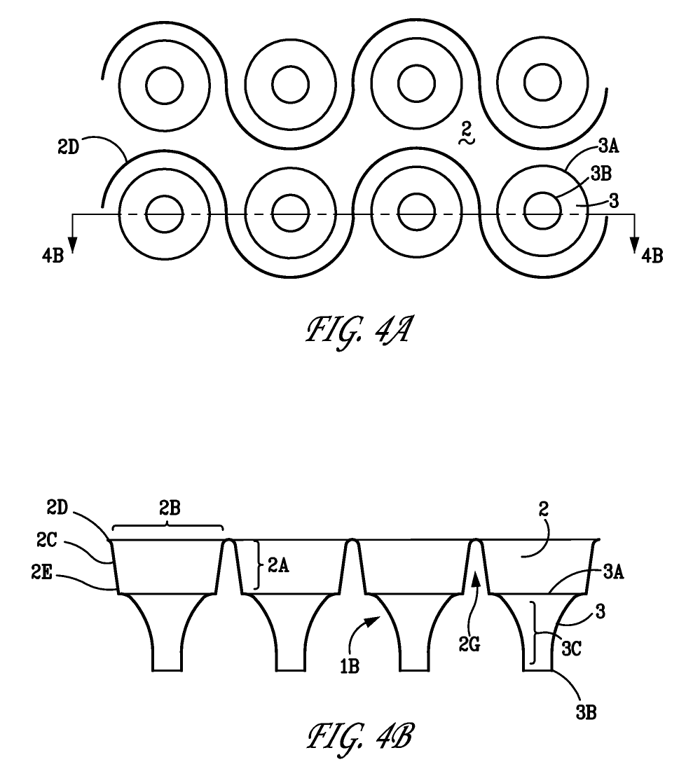

The top sheet 11 comprises at least novel apertured film 1 of this application and optionally a very thin, highly liquid pervious nonwoven layer 5. The apertured film 1 per this invention has a series of channels, conveniently side-by-side channels 2 on its surface 1A that faces the body side (FIGS. 2 and 3). The phrase “side-by-side” means that each channel is immediately adjacent another, with no intervening land space, i.e. the channels are disposed on either side of a channel wall 2C (discussed below) with no flat spots in the film intervening. Referring now to FIGS. 4A and 4B, the channels 2 have finite depth 2A and width 2B defined by the channel walls 2C. The aperture holes 3 are located below channels 2, such that the mouths 3A of the aperture holes which receive the body liquid exudate are located at the bottom of the channels, and in one embodiment the aperture holes are tapered, such that the exit ends 3B of the aperture holes 3 have smaller diameters than the mouths 3A. The depth 3C of the aperture hole 3 is also known as the length of the aperture capillary.

The length of the channels 2 is a multiple of the equivalent diameter of the channel cross section for predominant movement of liquid exudate into and through the aperture holes 3 under a pressure differential with respect to liquid exudate movement along the channel length under pressure differential, thus managing the run-off. Since air has much lower density and viscosity as compared to body liquid exudates like urine and blood, the channels 2 are large enough for air/moisture movement with ease, but small enough for controlled movement of the body liquid exudate along the channels.

Considering that viscosity of body exudates ranges from urine on the low side (about 1 centipoise) to menstrual fluid (about 20 centipoise) on the high side, the corresponding desired liquid exudate in-take times are from less than about 2 seconds to about 10 seconds, respectively. Assuming laminar flow through the channels under a pressure differential of about a centimeter of water head, it is desired to have a channel length to channel equivalent diameter (L/D) ratio of about 20. However, since liquid exudate moves into the aperture holes while following the torturous path along the channels, L/D as low as 10 is acceptable.

The vertical walls 2C of the channels 2 can be slightly inclined or slightly curved from top 2D to bottom 2E where the top (mouth 3A) of the aperture holes 3 are located. The height/depth 2A of the channels 2 is preferably no larger than the diameter of the aperture hole mouths 3A at the top (liquid exudate receiving side/mouth 3A of the aperture hole 3), preferably about half the diameter of aperture hole mouth 3A. Typical height of the channel walls 2C can range from about 0.5 mm to about 2 mm. The top most part 2D of the channel wall 2C can be flat or rounded having a finite radius/width for more comfort to the skin resting on the top sheet. “Equivalent diameter” is defined as 4×(cross sectional area of the channel divided by the wetted perimeter) for liquid exudate flow involved along the channel length. The length of the channels is at least ten times their equivalent diameters, and the depths of the channels are between about 0.25 to about 1 times, or even between about 0.5 to about 0.75 times the diameters of the apertures.

The channels can be straight along their lengths or curved, e.g. sinusoidal (FIG. 2) or half circles, or Z-shaped along their lengths for creating torturous paths for liquid exudate flowing through the channels along the surface of the apertured film. In any case, it can be advantageous if a series of channels are disposed in a side-by-side relationship on the surface of the apertured film. The series of channels can be continuous across the film, or can be intersected by another series of like channels, disposed at an angle to the first series of channels (FIG. 3), so as to terminate the continuity of the first series of channels, while allowing continuity of the flow of moisture/air along the channels from the first series to the adjacent series situated at an angle to the first series.

The length of the channels can be at least ten times their widths, or even at least twenty times their widths, and the depths of the channels can be between about 0.25 to 1 times the diameter of the apertures, or even between about 0.5 to 0.75 times the diameter of the apertures, as measured at the aperture mouth.

The termination points 2F (FIG. 3) of the channels 2 can also be partially blocked by each other, thus preventing direct straight line communication of liquid exudate in the channel to the outside environment, except channels at the edges of the absorbent article, e.g., the edges along waist and leg cuffs for a diaper.

The channels at the periphery of the personal care article can open directly to the outside environment for venting moisture and air from the space between the top sheet and the adjacent body skin, thus aiding in maintaining the humidity in the space close to equilibrium with the outside environment. If the top sheet is bonded to the outer moisture permeable layer 6 at the periphery of the personal care article, the top sheet can extend beyond the boundaries of the absorbent core to allow the channels to open into the space between the absorbent core and the liquid impervious, moisture permeable outer layer 6 and hence, allowing moisture/air in the channels to vent into that space and eventually escape to exterior environment by diffusing through the moisture permeable outer layer 6, and thus helping in lowering the humidity and temperature of the air in the space adjacent to the body skin.

The apertured film of this invention can have a thin layer of nonwoven fabric 5 on top, facing the body side. The nonwoven fabric 5 should be very open and highly liquid pervious, allowing liquid exudates to pass through to the apertured film 1 underneath with ease. Suitable nonwoven fabrics or webs include spunbonded webs, meltblown webs, air-laid webs, wet-laid webs, carded webs, hydroentangled webs and combinations of the above.

The thin nonwoven fabric 5 can also have channels for air flow (not shown), similar to the above described apertured film 1. Channels can be formed into the nonwoven webs by embossing the sheets with a channeled pattern. Channels for air flow can also be created by an adhesive used in adhering the apertured film to the nonwoven fabric. In order to do so, the adhesive so-placed should be deposited in a series of lines having finite lengths, widths, depths, shape and pattern, reflecting the desired channel configuration between the apertured film 1 and the nonwoven layer 5.

The nonwoven fabric and the film may be placed into a face-to-face relationship. The two components are preferably secured to each other in these latter embodiments. Suitable methods for securing the two components include, but are not limited to adhesives, fusion including heat bonding and/or pressure bonding, ultrasonics, and dynamic mechanical bonding.

In addition, the bottom surface 1B of the apertured film 1 in contact with the absorbent core can have hydrophobic surface characteristics (e.g., surface tension of the LLDPE or LDPE resin based film without the use of wetting agent in the base resin, resins commonly used in manufacturing the apertured film). The hydrophobic characteristic of the bottom surface of the apertured film can be helpful in reducing rewet by reducing potential wetting of the bottom surface 1B of the apertured film 1 in contact with the wet absorbent core 4 that has received and stored the liquid exudate. The top side of the apertured film 1 that receives the liquid exudate is hydrophilic for rapid transport of the liquid exudate through the aperture holes 3. The hydrophilic characteristic is most commonly achieved by the use of wetting agents (surfactants) in the base resin used in film manufacturing, or by treating a surface of the film with the wetting agent. Alternatively, hydrophilicity can be achieved by forming a two-layered film, wherein the top layer is formed from intrinsically wettable polymer, such as nylon copolymers, polyesters or the like.

The hydrophobic characteristic of bottom side 1B of the apertured film 1 that is in contact with the absorbent core 4 helps in preventing formation of the free liquid exudate layer between the bottom side 1B of the apertured film 1 and absorbent core 4 and thus, helps in maintaining channel like spaces 2G under the apertured film open and free of any liquid exudate. The channel like space 2G on the bottom side 1B of the apertured film 1 are simultaneously formed while forming channels 2 by raised walls 2C along the sides of aperture holes 3 on the top side of the film 1 during the apertured film formation process. The channel like space 2G free of liquid exudate on the bottom side 1B also helps in moisture and air movement and venting the moisture to the space underneath the moisture permeable (but liquid impervious) outer layer or directly to the outside environment in the same manner as channels 2 on the top side of the apertured film 1, thus helping in lowering the humidity and temperature of the environment in the vicinity of the skin covered by the absorbent article. Due to the hydrophobic characteristics of the film side 1B facing absorbent core 4 that receives and stores body liquid exudate, the likelihood of liquid accumulation underneath the apertured film 1 is greatly reduced and accordingly, back flow of liquid exudate from absorbent core 4 and through aperture holes 3 to body skin, phenomena commonly known as re-wet, is also greatly reduced.

The film having hydrophobic surface characteristics on one side (bottom side 1B of the apertured film 1) and hydrophilic characteristics on the top side (apertured film side facing the body skin) can be manufactured by conventional co-extrusion technology where a wetting agent is used in the base resin for one film layer while no wetting agent is used in base resin for the second layer during the bi-layer film formation. Typical base resins like LLDPE, LDPE, used in the films of interest here, are hydrophobic in nature.

The apertured film 1 having aperture holes 3 located at the base of the above described channels 2 per this invention can be made in a single step using conventional apertured film formation technology where film is passed over a roll having aperture holes. Aperture holes can be formed in the film by using pressure differential across the aperture holes covered by the film. A heat source such as hot gases and/or heating the film on the rolls prior to the aperture formation roll is also used as needed for aiding the aperture formation process. The aperture formation roll has specific engraved patterns for producing the apertured film with specific channels and aperture holes configurations per this invention as described above, so that the channels and aperture holes of the desired configuration are formed simultaneously in a single step on the same roll. The size and shape of the channels and holes on the roll are engineered for achieving the desired level and rate of extension of the film through channels and the holes during the channel and aperture formation process. The pattern and shape of the channels and holes on the rolls used in aperture formation is reflecting the finished size and shape of the channels and aperture holes at the base of channels. The shell of the roll can be porous to air flow facilitating suction of the film in to the engraved channels and forming the channels on the film.

Formation of channels on the film can also be facilitated by a female/male-type embossing rolls arrangement where the shell of the female roll has engraved channels and holes at the base of the engraved channels. The film gets embossed with channel pattern at the nip space between the embossing rolls. The embossed film exiting the nip stays on the female roll for an additional period such that formation of apertures at the base of the channels takes place due to pressure differential across the holes covered by the embossed film. A male embossing roll having a porous shell can also facilitate simultaneous formation of aperture holes right at the nip space by allowing suction of air through the porous male shell under a pressure differential across the holes on the female shell covered by the embossed film right at the nip space.

Aperture holes can also be created by conventional needle penetration into the film like technique as well.

The base resin selected in film is of specific melt index, softening and melting temperatures, stress/strain characteristics corresponding to the processing temperature and rate of aperture formation used in the aperture formation process. In addition, the base resin used in film formation imparts softness as well as resilience to excessive irreversible deformation of the channels under normal load.

Alternatively, the continuous channel 2 above the aperture holes 3 can be formed by overlapping adjacent circular holes of finite diameter and depth, where the diameter of each overlapping circular hole is larger than the aperture hole at the bottom.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

I claim:

1. An apertured film comprising a polymer film having a first series of channels, each having a length, width and depth, on at least one surface thereof and apertures having diameters at the bottoms of and along the length of said channels, said apertures extending through the thickness of the film, and wherein each aperture is disposed at the bottom of a channel.

2. The apertured film of claim 1, wherein the channels are straight.

3. The apertured film of claim 1, wherein the channels are intersected by at least a second series of channels running at angles to said first series of channels.

4. The apertured film of claim 1, wherein the channels are curved.

5. The apertured film of claim 4, wherein the channels are sinusoidal.

6. The apertured film of claim 1, wherein the length of the channels is at least ten times their equivalent diameters, and the depths of the channels are between about 0.25 to about 1 times the diameters of the apertures.

7. The apertured film of claim 1, wherein the length of the channels is at least twenty times their equivalent diameters, and the depths of the channels are between about 0.5 to about 0.75 times the diameters of the apertures.

8. The apertured film of claim 1, wherein the width of the channels is at least the same as the diameters of the apertures.

9. The apertured film of claim 1, wherein the channels are continuous.

10. The apertured film of claim 3, wherein both series of channels are continuous.

11. The apertured film of claim 1, wherein the channels are disposed side-by-side.

12. The apertured film of claim 1, wherein the first surface of said apertured polymer film is hydrophilic and the second surface is hydrophobic.

13. An absorbent personal care article comprising:
an apertured polymer film having a first series of channels, each having a length, width and depth, on a first surface thereof and apertures having diameters at the bottoms of and along the length of said channels, said apertures extending through the thickness of the film, wherein each aperture is disposed at the bottom of a channel, and through a second surface of said film; and
an absorbent core disposed against said second surface of said apertured film.

14. The absorbent personal care article of claim 13, further comprising a liquid pervious top layer of material, having first and second surfaces, and wherein said second surface is disposed against said first surface of said apertured polymer film.

15. The absorbent personal care article of claim 14, wherein said top layer material is a nonwoven material.

16. The absorbent personal care article of claim 15, wherein said nonwoven material is selected from the group consisting of a spunbonded webs, meltblown webs, air-laid webs, wet-laid webs, carded webs, hydroentangled webs and combinations thereof.

17. The absorbent personal care article of claim 14, wherein said second surface of said top layer is attached to said first surface of said apertured film.

18. The absorbent personal care article of claim 14, wherein said second surface of said top layer is attached to said first surface of said apertured film using an adhesive applied in a series of lines having finite spacing and depth, forming channels.

19. The absorbent personal care article of claim 13, further comprising a liquid impervious, moisture permeable back sheet disposed against said absorbent core opposite said apertured film.

20. The absorbent personal care article of claim 13, wherein the first surface of said apertured polymer film is hydrophilic and the second surface is hydrophobic.

21. The absorbent personal care article of claim 14, wherein said top layer material has a series of channels disposed on its second surface.

22. The absorbent personal care article of claim 13, which is selected from the group consisting of diapers, incontinence garments, training pants, catamenial pads, panty liners and bandages.

23. A process for forming an apertured film having channels on its surface and aperture holes at the bottom of said channels, comprising passing a film through a nip between male and female embossing rolls, wherein the female roll has engraved channels and apertures holes at the bottom of the engraved channels and the male embossing roll has raised wall patterns configured to correspond with said engraved channels.

24. The process of claim 23, wherein said film stays on the female embossing roll after passing through the nip and further comprises forming said aperture holes in the film by applying a pressure differential to the film at the sites of the aperture holes on the female embossing roll.

25. The process of claim 23, wherein the male embossing roll is porous to air flow under said pressure differential.
